(19) United States
(12) Patent Application Publication
(10) Pub. No.: US 2009/0047367 A1
(43) Pub. Date: Feb. 19, 2009

(54) Pharmaceutical composition for improving anxious behaviors of animals and method for preparing the same

(75) Inventors: Shuenn-Jyi Sheu, Taipei City (TW); Ching-Che Lin, Taipei City (TW); Hsiao-Yin Yeh, Taipei City (TW); Chen-Ying Chiang, Taipei City (TW)

Correspondence Address: BACON & THOMAS, PLLC, 625 SLATERS LANE, FOURTH FLOOR, ALEXANDRIA, VA, 22314-1176, US

(73) Assignee: Brion Research Institute of Taiwan, Taipei (TW)

(21) Appl. No.: 12/219,352

(22) Filed: Jul. 21, 2008

(30) Foreign Application Priority Data
Aug. 14, 2007 (TW) 096129957

(51) Int. Cl. A61K 33/10; A61K 33/06; A61K 36/899

(52) U.S. Cl. 424/687; 424/682; 424/750

(57) ABSTRACT

A pharmaceutical composition is disclosed in the present invention, which contains a medical material of Triticum aestivum L., a medical material of Glycyrrhizae Radix, and at least one calcium ion source. The pharmaceutical composition of the present invention can be taken by animals so as to improve anxious behaviors of the animals.

### BACKGROUND OF THE INVENTION

1. Field of the Invention

The present invention relates to a pharmaceutical composition and a method for preparing the same and, more particularly, to a pharmaceutical composition that can improve the anxious behaviors of animals and a method for preparing the same.

2. Description of Related Art

The symptoms of animal anxiety include: depression, loss of interests, psychomotor retardation, sleep disturbance, decrease in appetite, difficulty concentration, irritability, agitation, and even self-mutilation. Animals cannot express their depression by languages; hence there is some clinical evidence to show that these animals with anxiety bear repeating compulsive behaviors such as circling around, licking or biting their bodies (i.e. self-mutilation), destroying cages or plates, changing their excreting habits, cowering, barking or whining, having offensives action, and non-pathological abnormal molt. Some symptoms are even similar to manic-depressive psychosis of human beings.

Most anxious behaviors developing from domestic pets belong to separation anxiety. Separation anxiety is really a problem to pet owners, and it is a torment to pets and owners both. Separation anxiety can develop at any age or any period of feeding. Therefore, it is the most common reason for the pet owners to abandon their dogs or have them euthanized.

For example, dogs with separation anxiety will become extremely depressed while they are separated from their owners. However, the expressed behavior will usually vary among dogs. Some dogs will become very anxious once their owners disappear from their sight, so they may try to follow their owners around. Some dogs can endure being alone in rooms, but they may often survey whether their owners are home or not, and then they will feel at ease and play their toys or biting bones at other places. Some dogs may become anxious after their owners have left home. On the other hand, some dogs may be able to be left alone for a while when their owners have just left, but they will exhibit anxious behaviors if they are without human companionship for a long time.

Such symptoms of anxiety will increase as time goes by, and the expressed behavior is whining, pacing, slobbering, barking, howling, hyperactivity, scratching, chewing, digging, urinating, eating excrement, and destroying owners' things or furniture. Dogs with separation anxiety not only have one of above-mentioned behaviors, but also exhibit many behaviors at the same time. More severely, they even exhibit self-mutilation or escape by sometimes ruining windows and doors.

Dogs with separation anxiety display unusual excitement, such as barking, jumping at people, and circling for a long time when their owners come back, and they also exhibit such extraordinary excitement even though their owners have left home for just a few minutes.

The causes of separation anxiety remain unrealized currently. In addition, separation anxiety may take place whether there is just one pet or there are many pets at home. Some people believe that these pets are not well socialized in their childhood, and it means that in dogs without good interaction with humans or other dogs, their probability of developing separation anxiety will be increased. Besides, changes in owners' working times and family situations may also cause separation anxiety. Some dogs simply develop separation anxiety as they get older.

According to the research, although animals such as cats do not develop separation anxiety as easily as dogs, they may exhibit anxious behaviors when some situations occur such as owners moving to a new district.

Moreover, dogs which are held in cages for a long time and do not interact with human beings easily become spiritless, discouraged and depressed. This is just like imprisoned human beings easily experiencing mental problems. These problems cause a large influence on dogs' mental situations, and they may also cause the problems of immunity deficiency and other physical situations.

Currently, these problems of animal behavior are getting important, and Western societies use medicinal or non-medicinal methods to conduct psychology and ethology treatments in the light of pets with abnormal behaviors. Antidepressants including sedatives and pheromones are used mostly to conduct treatments. However, sedatives involve side effects that reduce the activity and sensitivity of ill pets, and pheromones are not easily available due to their expense. In addition, some specific pheromones act only on some specific animals and are not applied as widely as orally medications.

The major medicine presently used to treat anxious behaviors of animals is antidepressants used for humans. For example, fluoxetine (Prozac®) which is used mostly may cause some side effects, such as lethargy, anxiety, tremor, nausea, dyspepsia, abdominal discomfort, vasodilatation, dry mouth, sweating, skin rash, blurred vision, yawning, decrease in sex drive, and abnormal ejaculation. Moreover, the percentage of people who stop taking fluoxetine due to these related side effects is 15%, and it shows that these side effects are not low.

According to statistics, in clinical trials the most common events of fluoxetine occur in:

1. psychogenesis, mainly including nervousness, depression and insomnia;

2. digestive system, mainly including nausea which is the most frequent;

3. nervous system, mainly including vertigo;

4. body as a whole, mainly including weakness and headache; and

5. skin, mainly including skin rush and urticaria.

6. There are some reports showing that fluoxetine causes skin rash as the whole body and is involved in fatal events related to lung, kidney, and liver. However, whether it is caused by same/different etiology or pathology process is still unclear currently.

7. Rash occurrence or other unidentified etiologies may be caused by allergy.

Hence, if oral natural drugs, which can decrease the pet's anxious behavior without any side effects, are developed, it will bring large market value in the future.

In recent years, animal antianxiety researches using natural plants have been very rare, and only doctors of traditional Chinese medicine use Triticum aestivum L., Glycyrrhizae Radix, and Zizyphi Fructus as prescriptions to steady abnormal neural excitation and to eliminate rash convulsions. However, the effect of these prescriptions applied to treatment of animal anxiety is still unproved.

### SUMMARY OF THE INVENTION

According to the above-mentioned problems, the object of the present invention is to provide a pharmaceutical composition, and after animals have been treated with this pharmaceutical composition, their anxious behavior may be improved without experiencing any discomforting side effects.

The present invention discloses a pharmaceutical composition for improving anxious behaviors of animals, comprising: a medical material of Triticum aestivum L. with a content of 2˜48% by weight; a medical material of Glycyrrhizae Radix with a content of 2˜48% by weight; and at least one calcium ion source with a content of 4˜96% by weight.

The present invention also discloses a method for preparing a pharmaceutical composition for improving anxious behaviors of animals, comprising: providing a medical material of Triticum aestivum L., a medical material of Glycyrrhizae Radix, and at least one calcium ion source; mixing the medical material of Triticum aestivum L. and the medical material of Glycyrrhizae Radix to form a mixture; and adding the at least one calcium ion source into the mixture to form a pharmaceutical composition; wherein the content of the medical material of Triticum aestivum L. is 2˜48% by weight; the content of the medical material of Glycyrrhizae Radix is 2˜48% by weight; and the content of the medical material of the at least one calcium ion source is 4˜96% by weight.

The pharmaceutical composition and the method for preparing the same mentioned above may further comprise blood-nourishing plants selectively, and the blood-nourishing plants preferably are Zizyphi Fructus, and Lycii Fructus, wherein the content of the blood-nourishing plants is preferably 4˜96% by weight.

The medical material of Triticum aestivum L. is classified into unsaturated fruits of Triticum aestivum L. in the Gramineae family. In addition, the medical material of Triticum aestivum L. is preferably Floating Wheat, wherein it is obtained as follows: the general wheat is washed, and wheat floating on the water is named Floating Wheat.

The original sources of the medical material of Glycyrrhizae Radix are dry roots and subterranean stems of Glycyrrhizae uralensis Fisch and Glycyrrhizae glabra L., which belong to the Leguminosae family.

Additionally, the calcium ion source may comprise: an inorganic calcium salt, such as calcium carbonate, calcium phosphate, calcium acetate, and the derivatives thereof; a calcium-ligand chelate, such as a calcium amino acid chelate; dry bone (as a natural source of calcium phosphate) or bone glue of animals; and dry shell of shelled mollusks (also as outer shell or vestiges of inner shell), such as powders granulated from bivalves (and the common name of which is shellfish) by drying (as a natural source of calcium carbonate). The bivalvia belongs to the phylum Mollusk, and is named by its two-part shells. This class has 7500 species approximately, and the biggest size of which is 135 mm long. They include both marine and freshwater forms. Their heads have degenerated, and their feet are axe-shaped types. Additionally, they have a pair of gills on each side of their bodies. Their shells are situated at the sides of their bodies. The opening of their shells is passive, but the closing is completed by concentration of related muscles. They use their gills to catch food in the water by ciliary movement which causes whirlpools around their gills. They also get nutrient by mucous and tentacles through their mouths. Besides, many types of bivalves are the subjects for feeding and netting, just like pearl oysters.

In addition, the present invention also discloses a pharmaceutical composition for improving anxious behaviors of animals, comprising: a medical material of Zizyphi Fructus, a medical material of Triticum aestivum L., and a medical material of Glycyrrhizae Radix, wherein the content of each is 4˜96%, 2˜48%, and 2˜48%. The present invention further discloses that applying calcium ion source may also improve the anxious behaviors of animals.

The term “medical material” mentioned above refers to Chinese traditional herbs (including herbs of different plant basis but having the same efficacy, or common herbs used as substitutions), the extracts thereof, or the mixtures of the Chinese traditional herbs and the extracts thereof; and the extracts can be extracted by water, or alcohol.

Preferably, the pharmaceutical composition and the method for preparing the same in the present invention further comprise an excipient, and other pharmaceutically acceptably carriers if it is necessary. For example, they can be used in orally administered medical herb compositions, the formulations including: capsules, tablets, emulsifiers, liquid suspensions, dispersants, and solvents. Taking tablets as an illustration, the commonly used carriers are galactose or cornstarch; lubricants and magnesium stearate are basic additives. When oral capsules are used, galactose and dried cornstarch can act as effective diluting agents. Suitable sweetening agents, seasoning agents or pigments can be added if needed. The present invention may further comprise an excipient, which can be any kind known in the art but preferably is cornstarch.

Extracts in the pharmaceutical composition of the present invention can be prepared by conventional methods of preparation for scientific herbs, and preferably they are prepared by the method disclosed in the present invention. More preferably, the extracts are extracted by water, granulated by spray-drying, and then mixed proportionally with each other. In the case of calcium ion source, it may be prepared by decocting according to the conventional methods or by granulating to form powders, but more preferably by drying to form powders.

If the pharmaceutical composition of the present invention is administered to animals in an oral way, anxious behaviors of the animals such as separation anxiety can be efficiently improved.

### BRIEF DESCRIPTION OF THE DRAWINGS

FIG. 1 shows the comparison of CCR value between cats treated with drugs before and after in Example 4 of the present invention, and FIG. 2 shows the comparison of CCR value between dogs treated with drugs before and after in Example 4 of the present invention.

### DETAILED DESCRIPTION OF THE PREFERRED EMBODIMENT

By conventional methods of preparation for Scientifically Concentrated Traditional Chinese Medicines including extraction, separation, concentration, and dryness (as disclosed in Sun-Tien Medical Journal vols. 4 and 5, 2005), the extract powders of a medical material of Triticum aestivum L. and a medical material of Glycyrrhizae Radix were obtained and mixed with calcium ion source to form a pharmaceutical composition, and then administered to the animals with anxious behaviors in experiments.

The pharmaceutical composition (as prescription C) of the present invention, the prescription comprising Zizyphi Fructus, Floating Wheat, and Glycyrrhizae Radix (as prescription A) used in the treatment of human beings, and powders of shells and fried bones providing calcium ion (as prescription B), were used to compare the antianxiety effects in the present invention. The prescriptions A, B and C are shown as Table 1 as follows.

TABLE 1

| | | | | Calcium ion | | |
|---|---|---|---|---|---|---|
| | | Floating | Glycyrrhizae | Powders of | Powders of | Zizyphi |
| Example | Prescription | Wheat | Radix | shells | dried bones | Fructus |
| 1 | A | 65 | 20 | — | — | 15 |
| 2 | B | — | — | 50 | 50 | — |
| 3 | C | 40 | 20 | 40 | — | — |

Units are the weight percentage (%)

The animals with anxious behaviors were administered with each prescription mentioned above, and the conditions before and after administration were evaluated by their owners to identify the effect of each prescription. The dose administered to animals was 1.0 g/Kg for 2 weeks.

Herein, observing the phenomenon of animal anxiety is the most important aspect, for example, these animals with anxiety bear repeating compulsive behaviors such as circling around, licking or biting their bodies (i.e. self-mutilation), destroying cages or plates, changing their excreting habits, cowering, barking or whining, having offensive reaction, and non-pathological abnormal molt. According to the ill pets' behaviors observed by their owners, a standard as shown below was used to score (as 0 to 10 score based on the level of anxiety), and the owners recorded their pets' behaviors.
0 score: There is no phenomenon of anxious behaviors at all.
2 score: It seems that there is a trace phenomenon of anxious behaviors.
4 score: The owners were able to sense the anxious behaviors of their pets.
6 score: There is an apparent phenomenon of anxious behaviors, but it did not cause trouble to the owners.
8 score: There is an apparent phenomenon of anxious behaviors, and it caused trouble to the owners.
10 score: There is a very apparent phenomenon of anxious behaviors, and the owners had no choice but to separate their pets or to send them to veterinarians.

### EXAMPLE 1

### Prescription A

After the Sweet Wheat Dates soup (containing Glycyrrhizae Radix, Floating Wheat, and Glycyrrhizae Radix) which has been used in human beings in traditional Chinese medicine was administered to 20 animals with anxiety symptoms, the changes in anxious behaviors were observed, and the index of animal anxiety clinically recorded by owners is shown as Table 2 as follows.

TABLE 2

| | | | | | Index of | Index of | |
|---|---|---|---|---|---|---|---|
| | | | | | anxiety | anxiety | |
| | | | | | before | after | |
| | | | | | administering | administering | Observed phenomenon of |
| No. | Breed | Weight | Age | Gender | drugs | drugs | anxiety |
| 1 | Schnauzer | 3.5 | 5 | Male | 6 | 4 | It licked or bit feet to cause |
| | | | | | | | molt especially when owner |
| | | | | | | | was not home, and the |
| | | | | | | | frequency of licking its feet |
| | | | | | | | was decreased after |
| | | | | | | | administering drugs. |
| 2 | Golden | 33 | 1.2 | Male | 5 | 5 | It barked when owner was not |
| | Retriever | | | | | | home, and it didn't show any |
| | | | | | | | apparent effect after |
| | | | | | | | administering drugs. |
| 3 | Pomeranian | 3 | 3 | Female | 8 | 6 | It attacked other pets at home |
| | | | | | | | and became irritable, and its |
| | | | | | | | mood became more stable |
| | | | | | | | after administering drugs. |
| 4 | Yorkshire | 3 | 2 | Male | 7 | 5 | The barking efficiency was |
| | Terrier | | | | | | higher than usual and its |
| | | | | | | | appetite was decreased, and |
| | | | | | | | this case was a little better |
| | | | | | | | after administering drugs. |
| 5 | Shih Tzu | 4 | 1.5 | Female | 7 | 6 | It whined when separated |
| | | | | | | | from its owner, the frequency |
| | | | | | | | of whining was decreased |
| | | | | | | | after administering drugs. |
| 6 | Labrador | 29 | 5 | Male | 8 | 4 | It destroyed things and barked |
| | Retriever | | | | | | when its owner was not home, |
| | | | | | | | and this case was much better |
| | | | | | | | after administering drugs. |
| 7 | Maltese | 2.5 | 1.8 | Male | 7 | 5 | It became angry easily and |
| | | | | | | | attacked other dogs, and its |
| | | | | | | | mood became more stable |
| | | | | | | | after administering drugs. |
| 8 | Dachshund | 10 | 3 | Female | 7 | 5 | Its activity was decreased and |
| | | | | | | | it was in low spirit, and this |
| | | | | | | | case was slightly better after |
| | | | | | | | administering drugs. |
| 9 | Great Dane | 29 | 4 | Male | 7 | 4 | It destroyed things when its |
| | | | | | | | owner didn't come home for a |
| | | | | | | | long time, and this case was |
| | | | | | | | better after administering |
| | | | | | | | drugs. |
| 10 | Corgi | 10 | 3 | Female | 6 | 5 | It was anxious and bit feet |
| | | | | | | | when there were visitors, and |
| | | | | | | | the frequency of bitting its feet |
| | | | | | | | was decreased after |
| | | | | | | | administering drugs. |
| 11 | Red Poodle | 2 | 3 | Male | 6 | 5 | There were some situations in |
| | | | | | | | which it was too excited and |
| | | | | | | | was hardly controllable |
| | | | | | | | sometimes, and its mood |
| | | | | | | | became more stable after |
| | | | | | | | administering drugs. |
| 12 | Chinese | 8 | 6 | Male | 5 | 5 | It circled around repetitively, |
| | rural dog | | | | | | and this case did not show any |
| | | | | | | | apparent changes after |
| | | | | | | | administering drugs. |
| 13 | German | 35 | 5 | Male | 7 | 5 | It liked barking, and this case |
| | Shepherd | | | | | | became a little more |
| | Dog | | | | | | controllable after |
| | | | | | | | administering drugs. |
| 14 | Beagle | 10 | 2 | Female | 6 | 5 | There was a situation that it |
| | | | | | | | was hyperactive sometimes, |
| | | | | | | | and this case was better after |
| | | | | | | | administering drugs. |
| 15 | Siberian | 23 | 4 | Female | 6 | 4 | It destroyed things after its |
| | Huskies | | | | | | owner disciplined it, and its |
| | | | | | | | behavior was slightly |
| | | | | | | | improved after administering |
| | | | | | | | drugs. |
| 16 | Chihuahua | 2 | 2 | Female | 8 | 5 | It was too sensitive and it |
| | | | | | | | would bark if there were some |
| | | | | | | | noises. Its mood became a |
| | | | | | | | little bit more stable after |
| | | | | | | | administering drugs. |
| 17 | Domestic cat | 5 | 3 | Female | 8 | 4 | Its mood became unstable |
| | | | | | | | since moving house and there |
| | | | | | | | was a situation that it |
| | | | | | | | defecated everywhere. Its |
| | | | | | | | mood became stable in 5 days |
| | | | | | | | after administering drugs. |
| 18 | American | 5 | 5 | Male | 8 | 4 | It urinated arbitrarily |
| | Shorthair | | | | | | sometimes to attract its |
| | | | | | | | owners, and this case was |
| | | | | | | | happened rarely after |
| | | | | | | | administering drugs. |
| 19 | Persian cat | 4 | 10 | Female | 7 | 5 | Its activity was lowered and it |
| | | | | | | | interacted with its owner less. |
| | | | | | | | It slept in cat litter tray. After |
| | | | | | | | administering drugs, its spirit |
| | | | | | | | became better, and it would |
| | | | | | | | respond sometimes. |
| 20 | Domestic cat | 4 | 8 | Female | 8 | 5 | Its appetite and activity were |
| | | | | | | | lower after new pets arrived, |
| | | | | | | | and this case was slightly |
| | | | | | | | better after administering |
| | | | | | | | drugs. |

| | | |
|---|---|---|
| Average index of anxiety | 6.8 | 4.8 |

The unit of weight is kg, and the unit of age is year.

Before administering drugs, the average score of anxiety was 6.8. After administering drugs for two weeks, the average score of anxiety was decreased to 4.8. According to the changes in average score, there should be some improving effects to anxious behaviors of animals by administering Sweet Wheat Dates soup.

### EXAMPLE 2

### Prescription B

Using the calcium ion source as the experimental aim, the calcium ion source in this example was formed from bone of animals and shells of bivalves (and the common name of which is shellfish). After drying and granulating into powders, the calcium ion source was administered to 20 animals with anxiety symptoms, and then the changes in anxious behaviors were observed. The index of animal anxiety clinically recorded by owners is shown as Table 3 as follows.

TABLE 3

| | | | | | Index | | |
|---|---|---|---|---|---|---|---|
| | | | | | of | Index of | |
| | | | | | anxiety | anxiety | |
| | | | | | before | after | |
| | | | | | taking | taking | Observed phenomenon of |
| No. | Breed | Weight | Age | Gender | drugs | drugs | anxiety |
| 1 | Dachshund | 11 | 6 | Male | 6 | 3 | It refused to eat and drink when |
| | | | | | | | its owner was not home. It |
| | | | | | | | didn't refuse to eat after |
| | | | | | | | administering drugs. |
| 2 | Shih Tzu | 5 | 8 | Male | 7 | 5 | It urinated arbitrarily since |
| | | | | | | | moving house. The frequency |
| | | | | | | | of arbitrary urinating was |
| | | | | | | | reduced after administering |
| | | | | | | | drugs. |
| 3 | Red Poodle | 1.8 | 5 | Female | 7 | 5 | It did not eat well and vomited, |
| | | | | | | | and the situation about eating |
| | | | | | | | was improved after |
| | | | | | | | administering drugs. |
| 4 | Shetland | 20 | 3 | Female | 7 | 5 | It barked, whined and circled |
| | Sheepdog | | | | | | around when its owner was out, |
| | | | | | | | and the frequency of barking |
| | | | | | | | was reduced after |
| | | | | | | | administering drugs. |
| 5 | Papillon | 5 | 5 | Female | 7 | 5 | It liked to bark and scratch |
| | | | | | | | when it was out, and its owner |
| | | | | | | | was able to inhibit this behavior |
| | | | | | | | effectively after administering |
| | | | | | | | drugs. |
| 6 | Golden | 30 | 6 | Female | 6 | 6 | It bit things arbitrarily and |
| | Retriever | | | | | | made surroundings a mess, but |
| | | | | | | | this case was apparently not |
| | | | | | | | changed after administering |
| | | | | | | | drugs. |
| 7 | Domestic | 15 | 2 | Male | 7 | 5 | It bit its cage and barked at |
| | dog | | | | | | night, and its mood became |
| | | | | | | | more stable after administering |
| | | | | | | | drugs. |
| 8 | Chihuahua | 1.5 | 3 | Female | 8 | 6 | It bit other dogs and furniture, |
| | | | | | | | and its behavior was slightly |
| | | | | | | | improved after administering |
| | | | | | | | drugs. |
| 9 | Border | 15 | 3.5 | Female | 7 | 5 | It would refuse to eat and drink |
| | Collie | | | | | | unless its owner fed it, and this |
| | | | | | | | case was slightly better after |
| | | | | | | | administering drugs. |
| 10 | Maltese | 3 | 5.5 | Male | 8 | 5 | It refused to eat and drink |
| | | | | | | | unless its owner fed it, and it |
| | | | | | | | barked and whined when its |
| | | | | | | | owner was not in its vision. |
| | | | | | | | This case was a little bit |
| | | | | | | | improved after administering |
| | | | | | | | drugs. |
| 11 | Beagle | 11 | 9 | Female | 6 | 5 | It was restless and it whined |
| | | | | | | | sometimes, and its mood |
| | | | | | | | became a little bit more stable |
| | | | | | | | after administering drugs. |
| 12 | Schnauzer | 7 | 2 | Male | 8 | 5 | It would become angry and bite |
| | | | | | | | sofa if its owner touched it, and |
| | | | | | | | it started to close its owner |
| | | | | | | | after administering drugs. |
| 13 | Chow Chow | 30 | 10 | Male | 6 | 5 | The time it slept was longer |
| | | | | | | | than usual and its activity was |
| | | | | | | | reduced. This case was slightly |
| | | | | | | | better after administering |
| | | | | | | | drugs. |
| 14 | Shar Pei | 21 | 3 | Female | 8 | 7 | It bit feet and abdominal skin |
| | | | | | | | and whined sometimes, and the |
| | | | | | | | frequency that this case |
| | | | | | | | happened was reduced after |
| | | | | | | | administering drugs. |
| 15 | Yorkshire | 2.5 | 5 | Male | 7 | 5 | It hid in the dark and refused to |
| | Terrier | | | | | | come out, and the amount that |
| | | | | | | | it ate was reduced obviously. |
| | | | | | | | This case rarely happened after |
| | | | | | | | administering drugs. |
| 16 | Akita | 26 | 5 | Female | 5 | 5 | Its appetite was worse than |
| | | | | | | | usual, and it did not like to be |
| | | | | | | | close with its owner. It didn't |
| | | | | | | | show any obvious changes after |
| | | | | | | | administering drugs. |
| 17 | Pekingese | 6 | 2 | Male | 8 | 5 | It barked at people and it was |
| | | | | | | | offensive. After administering |
| | | | | | | | drugs, this case became better, |
| | | | | | | | but it would still whine. |
| 18 | Scottish Fold | 5 | 4 | Female | 7 | 5 | It licked its tail excessively, and |
| | | | | | | | the barking frequency is higher |
| | | | | | | | than before. The frequency of |
| | | | | | | | barking was reduced after |
| | | | | | | | administering drugs. |
| 19 | Domestic cat | 5 | 7 | Male | 8 | 4 | It hid, ate nothing, bit feet, and |
| | | | | | | | did not respond to its owner |
| | | | | | | | after shaving. After |
| | | | | | | | administering drugs, it started |
| | | | | | | | to close its owner. |
| 20 | American | 6 | 4 | Male | 7 | 5 | It was extremely excited when |
| | Shorthair | | | | | | its owner came home and |
| | | | | | | | accidentally injured its owner |
| | | | | | | | and other cats. Its mood |
| | | | | | | | became stable after |
| | | | | | | | administering drugs. |

| | | |
|---|---|---|
| Average index of anxiety | 7.0 | 5.1 |

The unit of weight is kg, and the unit of age is year.

Before administering drugs, the average score of anxiety was 7.0. After administering drugs for two weeks, the average score of anxiety was decreased to 5.1. According to the changes in average score, there should be some improving effects to anxious behaviors of animals by administering the calcium ion source, and the effect is close to the Sweet Wheat Dates soup.

### EXAMPLE 3

### Prescription C

Using the calcium ion source with Triticum aestivum L. and Glycyrrhizae Radix as the experimental aim, wherein the calcium ion source in this example was formed from bone of animals and shells of bivalves (and the common name of which is shellfish) by calcining and granulating into powders. The prescription C was administered to 20 animals with anxiety, and then the changes in anxious behaviors were observed. The index of animal anxiety clinically recorded by owners is shown as Table 4 as follows.

TABLE 4

| | | | | | Index of | Index of | |
|---|---|---|---|---|---|---|---|
| | | | | | anxiety | anxiety | |
| | | | | | before | after | |
| | | | | | administering | administering | Observed phenomenon of |
| No. | Breed | Weight | Age | Gender | drugs | drugs | anxiety |
| 1 | Fox Terrier | 7.5 | 4 | Male | 6 | 2 | Its appetite and activity were |
| | | | | | | | reduced both, and it did not |
| | | | | | | | respond when its owner |
| | | | | | | | called it. Its appetite |
| | | | | | | | recovered and it responded |
| | | | | | | | well after administering |
| | | | | | | | drugs. |
| 2 | Schnauzer | 6 | 4 | Female | 8 | 4 | It barked extremely, urinated |
| | | | | | | | arbitrarily, and destroyed |
| | | | | | | | things. This case was better |
| | | | | | | | and its mood became more |
| | | | | | | | stable after administering |
| | | | | | | | drugs. |
| 3 | Cocker | 40 | 4.5 | Female | 7 | 1 | It attacked new pets′ homes |
| | Spaniel | | | | | | and barked at its owner. This |
| | | | | | | | case improved a lot after |
| | | | | | | | taking drugs. |
| 4 | Chinchilla | 3.5 | 8 | Female | 5 | 5 | It was not spirited and it was |
| | | | | | | | rarely close to its owner. It |
| | | | | | | | did not show any obvious |
| | | | | | | | change after administering |
| | | | | | | | drugs. |
| 5 | Pomeranian | 2.5 | 2 | Male | 8 | 2 | It did not want to go out, and |
| | | | | | | | it would become scared and |
| | | | | | | | incontinent when it saw the |
| | | | | | | | same kinds. It became more |
| | | | | | | | regular after administering |
| | | | | | | | drugs. |
| 6 | Pug | 8 | 2 | Male | 6 | 2 | It refused to eat and drink |
| | | | | | | | when its owner was not |
| | | | | | | | home. It started to eat and |
| | | | | | | | drink after administering |
| | | | | | | | drugs. |
| 7 | Shih Tzu | 6 | 8 | Male | 7 | 1 | It barked and did not want to |
| | | | | | | | leave near the doorway when |
| | | | | | | | its owner was out, and it |
| | | | | | | | became more regular after |
| | | | | | | | taking drugs. |
| 8 | Shiba | 12 | 4 | Male | 7 | 1 | It bit things and barked |
| | | | | | | | arbitrarily, and its mood |
| | | | | | | | became more stable after |
| | | | | | | | administering drugs. |
| 9 | Maltese | 2 | 5 | Female | 5 | 2 | It followed its owner all the |
| | | | | | | | time and it would become |
| | | | | | | | uneasy when its owner was |
| | | | | | | | out its vision. This case |
| | | | | | | | became better after |
| | | | | | | | administering drugs. |
| 10 | Great | 37 | 4 | Male | 7 | 2 | It did not like to go out and it |
| | Pyrenean | | | | | | showed no response to its |
| | | | | | | | owner. This case was better |
| | | | | | | | and the interaction between it |
| | | | | | | | and its owner was getting |
| | | | | | | | more after administering |
| | | | | | | | drugs. |
| 11 | Yorkshire | 2 | 6 | Female | 8 | 3 | It hid itself and bit its |
| | Terrier | | | | | | forward feet, and it became |
| | | | | | | | more regular after |
| | | | | | | | administering drugs. |
| 12 | Domestic | 23 | 10 | Female | 7 | 2 | It would bark and snap its |
| | dog | | | | | | owner's things if its owner |
| | | | | | | | was going out. This |
| | | | | | | | phenomenon was reduced |
| | | | | | | | after administering drugs. |
| 13 | Norfolk | 5 | 4 | Female | 7 | 1 | It was offensive sometimes |
| | Terrier | | | | | | and it ate too much. The |
| | | | | | | | offensiveness was reduced |
| | | | | | | | and its appetite became |
| | | | | | | | normal after administering |
| | | | | | | | drugs. |
| 14 | Labrador | 28 | 2 | Female | 6 | 2 | It was spiritless and was not |
| | Retriever | | | | | | willing to eat. This case |
| | | | | | | | improved after administering |
| | | | | | | | drugs. |
| 15 | Beagle | 12 | 3 | Male | 7 | 3 | It did not pay attention to |
| | | | | | | | anyone, and this case became |
| | | | | | | | better administering taking |
| | | | | | | | drugs. |
| 16 | Golden | 34 | 4 | Male | 7 | 2 | It barked and bit things, and |
| | Retriever | | | | | | it was hard to control. It |
| | | | | | | | became obedient and its |
| | | | | | | | mood was more stable after |
| | | | | | | | administering drugs. |
| 17 | Saint | 55 | 3 | Female | 7 | 2 | The interaction with the same |
| | Bernard | | | | | | kinds was reduced, and the |
| | | | | | | | time it slept was longer than |
| | | | | | | | usual. Besides, it was |
| | | | | | | | spiritless. Its spirit became |
| | | | | | | | better and it had more vitality |
| | | | | | | | after administering drugs. |
| 18 | Siamese | 4 | 6 | Male | 7 | 1 | It would whine for a long |
| | | | | | | | time if it was shaved |
| | | | | | | | excessively. This case |
| | | | | | | | improved after administering |
| | | | | | | | drugs. |
| 19 | Russian | 4 | 6 | Male | 8 | 4 | There were some situations |
| | Blue | | | | | | of urinating arbitrarily and |
| | | | | | | | sleep disorder after the house |
| | | | | | | | was redecorated. Its sleeping |
| | | | | | | | condition became more |
| | | | | | | | normal after administering |
| | | | | | | | drugs. |
| 20 | American | 4.5 | 8 | Female | 8 | 2 | It pulled out its hair and it |
| | shorthair | | | | | | urinated arbitrarily. This |
| | | | | | | | condition was obviously |
| | | | | | | | improved after administering |
| | | | | | | | drugs. |

| | | |
|---|---|---|
| Average index of anxiety | 6.9 | 2.2 |

The unit of weight is kg, and the unit of age is year.

Before administering drugs, the average score of anxiety was 6.9 After administering drugs for two weeks, the average score of anxiety was decreased to 2.2. According to the changes in average score, there should be some improving effects to anxious behaviors of animals obviously by administering the prescription C, and the effect is much better than using the Sweet Wheat Dates soup or the calcium ion source alone. Hence, using Triticum aestivum L. and Glycyrrhizae Radix with the calcium ion source together has the addictive effect. Therefore, this prescription bears inventive step.

### EXAMPLE 4

### Prescription C

In order to identify the effect of prescription C administered to the anxious animals, thus the method of urinary analysis was used, which was published by Anthrozoology Institute at University of Bristol (Hiby E. F. et al., 2006, Behavioral and physiological responses of dogs entering re-homing kennels, Physiology & Behavior, 89(3): 385-391), to examine the variations in cortisol/creatinine ratio (CCR). The variations in CCR value can be used as a direct evidence for the stress of animals (i.e. anxiety). If there are specific statistics differences in the variation in CCR value after the experimental animals taking these prescriptions in the present invention, it means that the prescriptions in the present invention indeed improve the anxious behaviors of animals.

In the experiments according to the method, the most suitable time to take urinate for analyzing the CCR value is in the early morning, i.e. the first urinate after awaking and preferably the mid stream urine. The analysis shows that the dogs with lower CCR value in urine samples are more easily bred. It means that these dogs may adapt to environment and accompany humans easily.

Therefore, the prescription C was administered to the ill pets which are 5 cats and 4 dogs, and the changes in anxious behaviors were observed. The clinical observation of anxious behaviors was recorded and shown in Table 5 as follows.

TABLE 5

| No. | Breed | Weight | Age | Gender | Observed phenomenon of anxiety |
|---|---|---|---|---|---|
| 1 | Domestic | 12 | 3-4 | Male | 2007/3/05(Before administration): |
| | dog | | | | It licked feet and its thenars were red-brown. It |
| | | | | | interacted with its owner badly except eating. |
| | | | | | 2007/3/18(After administration): |
| | | | | | This case improved from the 7^{th} day and |
| | | | | | maintained the improved state in the whole |
| | | | | | period of administration. |
| 2 | Domestic | 10 | 1 | Female | 2007/3/06(Before administration): |
| | dog | | | | It was offensive and barked without any reasons. |
| | | | | | (It had been a stray dog) |
| | | | | | 2007/3/19(After administration): |
| | | | | | The frequency of barking was reduced from the |
| | | | | | 7^{th} day, but the anxious phenomenon was |
| | | | | | recovered after stopping administration for 3 |
| | | | | | days. |
| 3 | Maltese | 4.8 | 5 | Male | 2007/3/02(Before administration): |
| | | | | | It licked feet severely and was not able to be |
| | | | | | controlled by punishment, and thus it had got |
| | | | | | dactylitis. |
| | | | | | 2007/3/15(After administration): |
| | | | | | The frequency of licking feet was decreased from |
| | | | | | the 6^{th} day, but it would still lick feet if it was |
| | | | | | nervous. |
| 4 | Huskies | 20 | 4 | Male | 2007/3/24(Before administration): |
| | | | | | It was restless and it played excessively. It would |
| | | | | | jump at people and eat fussily if it was too |
| | | | | | excited. |
| | | | | | 2007/4/06(After administration): |
| | | | | | It became stable from the 2^{nd} day. It would be |
| | | | | | tender as long as being administered drugs. Its |
| | | | | | excrements were very hard. |
| 5 | American | 6 | 2.5 | Female | 2007/2/05(Before administration): |
| | Shorthair | | | | It urinated arbitrarily, and there was molt at the |
| | | | | | back of its neck. |
| | | | | | 2007/2/18(After administration): |
| | | | | | It did not urinate arbitrarily on the 7^{th}, 8^{th} and 11^{th} |
| | | | | | day, but molt at the back of its neck still didn't be |
| | | | | | mended. |
| 6 | Domestic cat | 4.5 | 2.5 | Female | 2007/3/10(Before administration): |
| | | | | | It urinated arbitrarily and it chased and attacked |
| | | | | | the new cats at home. |
| | | | | | 2007/3/26(After administration): |
| | | | | | The frequency of urinating arbitrarily was |
| | | | | | reduced for the whole, and the situation which it |
| | | | | | chased cats was improved after administration. |
| 7 | Domestic cat | 3.2 | 4 | Female | 2007/3/08(Before administration): |
| | | | | | It licked its belly and forward feet to lose all the |
| | | | | | hairs. The hairs on its back were molting. It |
| | | | | | would be excited easily when it saw the |
| | | | | | strangers. |
| | | | | | 2007/3/22(After administration): |
| | | | | | Its mood became stable and it followed people |
| | | | | | from the 5^{th} day. There were new hairs grown in |
| | | | | | the belly and back. |
| 8 | Chinchilla | 3 | 3 | Female | 2007/3/19(Before administration): |
| | | | | | It was shy to the strangers, and it did not like |
| | | | | | people hugging it. |
| | | | | | 2007/4/01(After administration): |
| | | | | | It started following people and it would sleep |
| | | | | | besides its owner. |
| 9 | Domestic cat | 6 | 6 | Male | 2007/3/20(Before administration): |
| | | | | | The dog moved to a new house and became very |
| | | | | | unfriendly. It attached actively. It was not close to |
| | | | | | its owner. It cried and barked all the nights. |
| | | | | | Besides, its owner was not able to let it eat |
| | | | | | powders but forced it to eat caplets. |
| | | | | | 2007/4/09(After administration): |
| | | | | | It was not scared of other dogs from the 5^{th} day. It |
| | | | | | was unhappy sometimes but could be touched |
| | | | | | closely. Also, it interacted with its owner. |
| | | | | | Its active time was the same as the time before |
| | | | | | moving house from the 6^{th} day, and it also slept |
| | | | | | on its owner's body. |
| | | | | | It was willing to eat powders with can food from |
| | | | | | the 7^{th} day. Also, it barked at other dogs |
| | | | | | sometimes but was not willing to do this. |

The unit of weight is kg, and the unit of age is year.

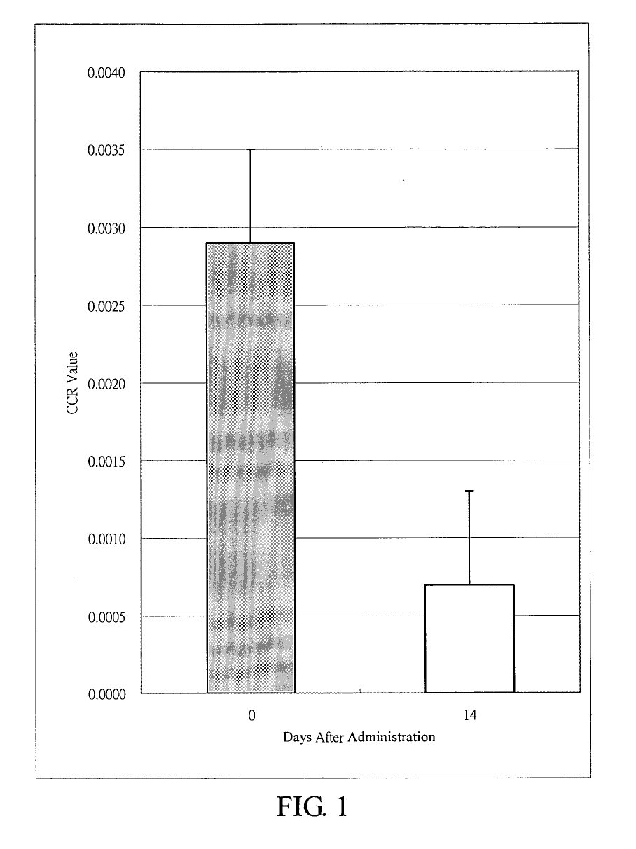

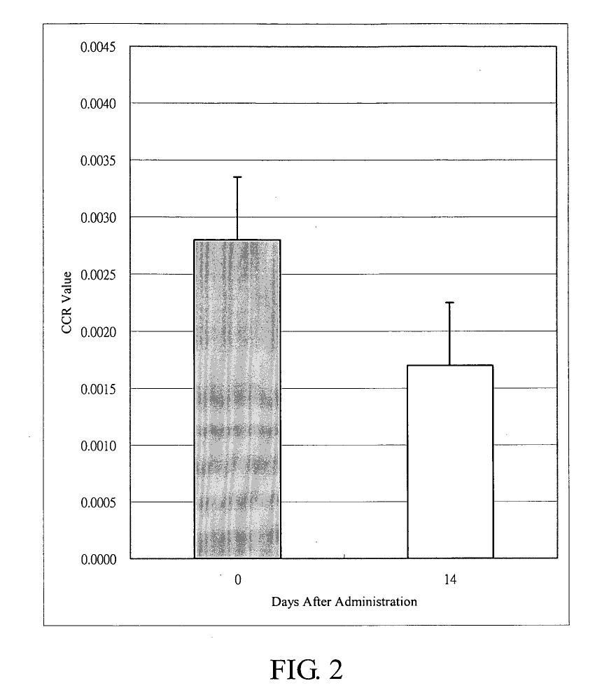

The CCR value in accordance with the urine of the ill animals was also analyzed. The urine for analysis was obtained from the mid stream urine of the 1^{st} urine in the early morning, which was taken from the ill pets before and after administered for 14 days. FIG. 1 is the variation in CCR value before and after administering to the No. 5˜9 ill cats in Table 5. As shown as FIG. 1, it is seen that the average value of CCR of ill cats before administration was 0.0029 (SD=0.0006), and the average value of CCR was reduced to 0.0007 (SD=0.0006) after administration. FIG. 2 is the variation in CCR value before and after administering to the No. 1˜4 ill dogs in Table 5. As shown as FIG. 2, it is seen that the average value of CCR of ill dogs before administration was 0.0028 (SD=0.0006), and the average value of CCR was reduced to 0.0017 (SD=0.0005) after administration. After the two group data mentioned above were analyzed by statistical software to complete T test, the p values all were lower than 0.05, and it indicated that the variation in CCR value before and after administering to dogs and cats was quite significant. From the variation in CCR value (i.e. index of pressure) in this example, the effect of the pharmaceutical composition in the present invention is proven. According to the experimental results mentioned above, it is sufficient to verify that the anxious behaviors of animals may be improved significantly by using the pharmaceutical in the present invention. Hence, the present invention bears inventive step.

Although the present invention has been explained in relation to its preferred embodiment, it is to be understood that many other possible modifications and variations can be made without departing from the scope of the invention as hereinafter claimed.

What is claimed is:

1. A pharmaceutical composition for improving anxious behaviors of animals, comprising:
a medical material of Triticum aestivum L. with a content of 2˜48% by weight;
a medical material of Glycyrrhizae Radix with a content of 2˜48% by weight; and
at least one calcium ion source with a content of 4˜96% by weight.

2. The pharmaceutical composition as claimed in claim 1, wherein the medical material of Triticum aestivum L. is Floating Wheat.

3. The pharmaceutical composition as claimed in claim 1, wherein these medical materials are plants, extracts of plants, or combinations thereof.

4. The pharmaceutical composition as claimed in claim 3, wherein the extracts of plants are extracted by water, or alcohol.

5. The pharmaceutical composition as claimed in claim 1, wherein the calcium ion sources is an inorganic calcium salt, or an organic calcium salt.

6. The pharmaceutical composition as claimed in claim 5, wherein the inorganic calcium salt is calcium carbonate, calcium phosphate, calcium acetate, or the derivatives thereof.

7. The pharmaceutical composition as claimed in claim 5, wherein the organic calcium salt is a calcium-ligand chelate, dry bone or bone glue of animals, or dry shell of shelled mollusks.

8. The pharmaceutical composition as claimed in claim 7, wherein the calcium-ligand chelate is a calcium amino acid chelate.

9. The pharmaceutical composition as claimed in claim 7, wherein the dry shell of shelled mollusks is dried inner or outer shell of mollusks bivalvias.

10. The pharmaceutical composition as claimed in claim 1, further comprising a blood-nourishing plant.

11. The pharmaceutical composition as claimed in claim 10, wherein the blood-nourishing plants are Zizyphi Fructus, and Lycii Fructus.

12. The pharmaceutical composition as claimed in claim 10, wherein the blood-nourishing plant is Zizyphi Fructus.

13. The pharmaceutical composition as claimed in claim 1, which is used to treat anxious behaviors of animals.

14. A method for preparing a pharmaceutical composition for improving anxious behaviors of animals, comprising:
providing a medical material of Triticum aestivum L., a medical material of Glycyrrhizae Radix, and at least one calcium ion source;
mixing the medical material of Triticum aestivum L. and the medical material of Glycyrrhizae Radix to form a mixture; and
adding the at least one calcium ion source into the mixture to form a pharmaceutical composition;
wherein the content of the medical material of Triticum aestivum L. is 2˜48% by weight; the content of the medical material of Glycyrrhizae Radix is 2˜48% by weight; and the content of the medical material of the at least one calcium ion source is 4˜96% by weight.

15. The method as claimed in claim 14, wherein the medical material of Triticum aestivum L. is a medical material of Floating Wheat.

16. The method as claimed in claim 14, wherein these medical materials are plants, extracts of plants, or combinations thereof.

17. The method as claimed in claim 16, wherein the extracts of plants are extracted by water, or alcohol.

18. The pharmaceutical composition as claimed in claim 17, further comprising powders granulated from the extracts of medical materials by spray-drying, or freeze-drying.

19. The method as claimed in claim 14, wherein the calcium ion source is an inorganic calcium salt, or an organic calcium salt.

20. The method as claimed in claim 19, wherein the inorganic calcium salt is calcium carbonate, calcium phosphate, calcium acetate, and the derivatives thereof.

21. The method as claimed in claim 19, wherein the organic calcium salt is a calcium-ligand chelate, dry bone or bone glue of animals, or dry shell of shelled mollusks.

22. The method as claimed in claim 21, wherein the calcium-ligand chelate is a calcium amino acid chelate.

23. The method as claimed in claim 22, wherein the dry shell of shelled mollusks is dried inner or outer shell of mollusks bivalvias.

24. The method as claimed in claim 14, further comprising a blood-nourishing plant, which is combined with the pharmaceutical composition.

25. The method as claimed in claim 24, wherein the blood-nourishing plants are Zizyphi Fructus and Lycii Fructus.

26. The method as claimed in claim 24, wherein the blood-nourishing plant is Zizyphi Fructus.

27. The method as claimed in claim 14, further comprising an excipient, and mixing the excipient with the pharmaceutical composition.
